(19) United States
(12) Patent Application Publication
(10) Pub. No.: US 2007/0006395 A1
(43) Pub. Date: Jan. 11, 2007

(54) Agents containing 4-(2-hydroxyethyl)amino-3-nitro-1-benzotrifluoride for dyeing keratin fibers

(76) Inventors: Hansruedi Muerner, St Ursen (CH); Herbert Mager, Marly (CH)

Correspondence Address: Striker Striker & Stenby, 103 East Neck Road, Huntington, NY, 11743, US

(21) Appl. No.: 10/545,635

(22) PCT Filed: Oct. 19, 2004
(86) PCT No.: PCT/EP04/11791

§ 371 Date: May 26, 2006

(30) Foreign Application Priority Data
Jan. 30, 2004 (DE) 102004004733.2

(51) Int. Cl. A61K 8/00

(52) U.S. Cl. 8/405

(57) ABSTRACT

The composition for coloring keratin fibers contains (a) 4-(2′-hydroxyethyl)amino-3-nitro-1-trifluoromethylbenzene or a salt thereof, (b) one or more developers and (c) one or more couplers. In the method for coloring hair the colorant composition is mixed with an oxidant to form a ready-to-apply colorant and then the ready-to-apply colorant is applied to hair in an amount sufficient for the dyeing of the hair.

The present invention concerns 4-(2-hydroxyethyl)amino-3-nitro-1-trifluoromethyl-benzene-containing agents for the oxidative dyeing of keratin fibers, particularly human hair.

In the field of dyeing keratin fibers, particularly hair dyeing, oxidation dyes have attained substantial importance. In this case, the color is produced by reaction of certain developers with certain couplers in the presence of an appropriate oxidant. Suitable developers for this purpose are, in particular, 2,5-diaminotoluene, 2,5-diaminophenylethyl alcohol, p-aminophenol, 1,4-diaminobenzene and 4,5-diaminopyrazole-1-(2-hydroxyethyl) [sic], whereas suitable couplers are, for example, resorcinol, 2-methylresorcinol, 1-naphthol, 3-aminophenol, m-phenylenediamine, 2-amino-4-(2′-hydroxyethyl)aminoanisole, 1,3-diamino-4-(2′-hydroxyethoxy)benzene, 2,4-diamino-5-fluorotoluene, 5-amino-2-methylphenol, 5-(2′-hydroxyethylamino)-2-methylphenol and N-(2′-hydroxyethyl)-3,4-methylenedioxyaniline.

In addition, in oxidative colorants, different direct dyes, particularly from the class of nitro dyes, are used as shading aids. Examples can be found on page 317 of the standard publication “Chemical and Physical Behavior of Human Hair” by Clarence R. Robbins (fourth edition, Springer 2002) and in EP 0 182 330 A2, among others. Nitro dyes, being small uncharged molecules, can readily penetrate into hair, but, as a rule, because of their size and lacking charge, are also readily washed out. In general, nitro dyes are poorly absorbed into long porous hair and hair tips and give unbalanced coloring results. Another drawback of nearly all nitro dyes is that they color the skin very strongly and that the wet, dyed hair shows a pronounced dye loss when rubbed. This loss on rubbing occurs even after the hair has been shampooed several times. Because of these properties, the use of nitro dyes in oxidation dyes, as a rule, is limited to low concentrations and/or mixed shades.

In addition to being expected to produce colorations of the desired intensity, oxidation dyes and nitro dyes intended for coloring human hair are subject to many additional requirements. Thus, in particular, these dyes must be toxicologically and dermatologically harmless. This requirement is not met by all dyes. For example, for 4-(1,2-propane-diol)amino-3-nitro-1-trifluoromethylbenzene and 4-(2′-hydroxyethyl)amino-3-nitro-1-chlorobenzene it is questionable whether these compounds meet the said requirements in every respect.

Hence, a great need existed for direct dyes that are suitable for use in oxidation hair colorants, give uniform and intense colorations and also are toxicologically and dermatologically harmless.

We have now found that the nitro dye 4-(2-hydroxyethyl)amino-3-nitro-1-trifluoromethylbenzene meets these requirements to a particularly high degree. 4-(2′-hydroxyethyl)amino-3-nitro-1-trifluoromethylbenzene [sic] dyes very well both the porous long hair and the slightly damaged hair tips imparting a uniform coloration to both damaged and undamaged hair. Moreover, following the dyeing, rubbing causes no wet dye loss and the skin is not colored. These properties result in permanence of the coloration on the hair which is comparable to that imparted by oxidation dyes resulting from conventional developers and couplers. This provides an outstanding possibility for combination with oxidation dyes and particularly with the developers and couplers more closely explained in the examples. Unexpectedly, the toxicological data turned out to be substantially more favorable than for the practically analogous compounds 4-(1,2-propanediol)amino-3-nitro-1-trifluoromethylbenzene and 4-(2-hydroxyethyl)amino-3-nitro-1-chlorobenzene.

Hence, the object of the present invention is an agent for coloring keratin fibers, particularly hair, characterized in that it contains a) 4-(2′-hydroxyethyl)amino-3-nitro-1-trifluoromethylbenzene or a salt thereof, b) one or more developers and c) one or more couplers.

4-(2′-Hydroxyethyl)amino-3-nitro-1-trifluoromethylbenzene or a salt thereof is contained in the colorant of the invention in an amount (based on the total amount of the ready-to-use colorant) from about 0.01 to 5 weight percent, preferably from about 0.01 to 3 weight percent and particularly from 0.1 to 1.5 weight percent (in each case based on the ready-to-use colorant).

Suitable developers are 1,4-diaminobenzene (p-phenylenediamine), 1,4-diamino-2-methylbenzene (p-toluylenediamine), 1,4-diamino-2,6-dimethylbenzene, 1,4-diamino-3,5-diethylbenzene, 1,4-diamino-2,5-dimethylbenzene, 1,4-diamino-2,3-dimethylbenzene, 2-chloro-1,4-diaminobenzene, 1,4-diamino-2-(thiophen-2-yl)benzene, 1,4-diamino-2-(thiophen-3-yl)benzene, 1,4-diamino-2-(pyridin-3-yl)benzene, 2,5-diaminobiphenyl, 1,4-diamino-2-methoxymethylbenzene, 1,4-diamino-2-aminomethylbenzene, 1,4-diamino-2-hydroxymethylbenzene, 1,4-diamino-2-(2-hydroxyethoxy)benzene, 2-[2-(acetylamino)ethoxy]-1,4-diaminobenzene, 4-phenylaminoaniline, 4-dimethylaminoaniline, 4-diethylaminoaniline, 4-dipropylaminoaniline, 4-[ethyl-(2-hydroxyethyl)amino]aniline, 4-[di(2-hydroxyethyl)-amino]aniline, 4-[di(2-hydroxyethyl)amino]-2-methylaniline, 4-[(2-methoxyethyl)amino]-aniline, 4-[(3-hydroxypropyl)amino]aniline, 4-[(2,3-dihydroxypropyl)amino]aniline, 1,4-diamino-2-(1-hydroxyethyl)benzene, 1,4-diamino-2-(2-hydroxyethyl)benzene, 1,4-diamino-2-(1-methylethyl)benzene, 1,3-bis[(4-aminophenyl)-(2-hydroxyethyl)amino]-2-propanol, 1,4-bis[(4-aminophenyl)amino]butane, 1,8-bis(2,5-diaminophenoxy)-3,6-dioxaoctane, 4-aminophenol, 4-amino-3-methylphenol, 4-amino-3-(hydroxymethyl)phenol, 4-amino-3-fluorophenol, 4-methylaminophenol, 4-amino-2-(aminomethyl)phenol, 4-amino-2-(hydroxymethyl)phenol, 4-amino-2-fluorophenol, 4-amino-2-[(2-hydroxyethyl)amino]methylphenol, 4-amino-2-methylphenol, 4-amino-2-(methoxymethyl)phenol, 4-amino-2-(2-hydroxyethyl)-phenol, 5-aminosalicylic acid, 2,5-diaminopyridine, 2,4,5,6-tetraaminopyrimidine, 2,5,6-triamino-4-(1H)-pyrimidone, 4,5-diamino-1-(2-hydroxyethyl)-1H-pyrazole, 4,5-diamino-1-(1-methylethyl)-1H-pyrazole, 4,5-diamino-1-[(4-methylphenyl)methyl]-1H-pyrazole, 1-[(4-chlorophenyl)methyl]-4,5-diamino-1H-pyrazole, 4,5-diamino-1-methyl-1H-pyrazole, 2-aminophenol, 2-amino-6-methylphenol, 2-amino-5-methylphenol and 1,2,4-trihydroxybenzene, among which 1,4-diamino-2-methylbenzene, 4,5-diamino-1-(2′-hydroxyethyl)-1H-pyrazole, 4-aminophenol and 1,4-(2,2′-hydroxyethyl)diaminobenzene [sic] are preferred and 4,5-diamino-1-(2′-hydroxyethyl)-1H-pyrazole and 2-(2′-hydroxyethyl)-1,4-diaminobenzene are particularly preferred.

Suitable couplers are, in particular, N-(3-dimethylaminophenyl)urea, 2,6-diaminopyridine, 2-amino-4-[(2-hydroxyethyl)amino]anisole, 2,4-diamino-1-fluoro-5-methylbenzene, 2,4-diamino-1-methoxy-5-methylbenzene, 2,4-diamino-1-ethoxy-5-methylbenzene, 2,4-diamino-1-(2-hydroxyethoxy)-5-methylbenzene, 2,4-di[(2-hydroxyethyl)amino]-1,5-dimethoxybenzene, 2,3-diamino-6-methoxypyridine, 3-amino-6-methoxy-2-(methylamino)pyridine, 2,6-diamino-3,5-dimethoxypyridine, 3,5-diamino-2,6-dimethoxypyridine, 1,3-diaminobenzene, 2,4-diamino-1-(2-hydroxyethoxy)benzene, 1,3-diamino-4-(2,3-dihydroxypropoxy)-benzene, 1,3-diamino-4-(3-hydroxypropoxy)benzene, 1,3-diamino-4-(2-methoxyethoxy)-benzene, 2,4-diamino-1,5-di(2-hydroxyethoxy)benzene, 1-(2-aminoethoxy)-2,4-diaminobenzene, 2-amino-1-(2-hydroxyethoxy)-4-methylaminobenzene, 2,4-diaminophenoxyacetic acid, 3-[di(2-hydroxyethyl)amino]aniline, 4-amino-2-di[(2-hydroxyethyl)amino]-1-eth-oxybenzene, 5-methyl-2-(1-methylethyl)phenol, 3-[(2-hydroxyethyl)amino]aniline, 3-[(2-aminoethyl)amino]aniline, 1,3-di(2,4-diaminophenoxy)propane, di(2,4-diaminophenoxy)-methane, 1,3-diamino-2,4-dimethoxybenzene, 2,6-bis(2-hydroxyethyl)aminotoluene, 4-hydroxyindole, 3-dimethylaminophenol, 3-diethylaminophenol, 5-amino-2-methylphenol, 5-amino-4-fluoro-2-methylphenol, 5-amino-4-methoxy-2-methylphenol, 5-amino-4-ethoxy-2-methylphenol, 3-amino-2,4-dichlorophenol, 5-amino-2,4-dichlorophenol, 3-amino-2-methylphenol, 3-amino-2-chloro-6-methylphenol, 3-aminophenol, 2-[(3-hydroxyphenyl)-amino]acetamide, 5-[(2-hydroxyethyl)amino]-4-methoxy-2-methylphenol, 5-[(2-hydroxy-ethyl)amino]-2-methylphenol, 3-[(2-hydroxyethyl)amino]phenol, 3-[(2-methoxyethyl)ami-no]phenol, 5-amino-2-ethylphenol, 5-amino-2-methoxyphenol, 2-(4-amino-2-hydroxy-phenoxy)ethanol, 5-[(3-hydroxypropyl)amino]-2-methylphenol, 3-[(2,3-dihydroxypropyl)-amino]-2-methylphenol, 3-[(2-hydroxyethyl)amino]-2-methylphenol, 2-amino-3-hydroxypy-ridine, 2,6-dihydroxy-3,4-dimethylpyridine, 5-amino-4-chloro-2-methylphenol, 1-naphthol, 2-methyl-1-naphthol, 1,5-dihydroxynaphthalene, 1,7-dihydroxynaphthalene, 2,3-dihydroxynaphthalene, 2,7-dihydroxynaphthalene, 2-methyl-1-naphthol acetate, (m-dihydroxyphenyl)acrylamide, 1-chloro-2,4-dihydroxybenzene, 2-chloro-(m-dihydroxyphenyl)acrylamide, 1,2-dichloro-3,5-dihydroxy-4-methylbenzene, 1,5-dichloro-2,4-dihydroxybenzene, 1,3-dihydroxy-2-methylbenzene, 3,4-methylenedioxyphenol, 3,4-methylenedioxyaniline, 5-[(2-hydroxyethyl)amino]-1,3-benzodioxole, 6-bromo-1-hydroxy-3,4-methylenedioxybenzene, 3,4-diaminobenzoic acid, 3,4-dihydro-6-hydroxy-1,4(2H)-benzoxazine, 6-amino-3,4-dihydro-1,4(2H)-benzoxazine, 3-methyl-1-phenyl-5-pyrazolone, 5,6-dihydroxyindole, 5,6-dihydroxyindoline, 5-hydroxyindole, 6-hydroxyindole, 7-hydroxyindole and 2,3-indolinedione, among which 1,3-dihydroxybenzene, 3-aminophenol, 5-amino-2-methylphenol, 1-naphthol, 1,3-diamino-4-(2′-hydroxyethoxy)benzene and N-(2′-hydroxyethyl)-3,4-methyl-enedioxyaniline are preferred and 5-amino-2-methylphenol, 1-naphthol, 1,3-diamino-4-(2′-hydroxyethoxy)benzene and N-(2′-hydroxyethyl)-3,4-methylenedioxyaniline are particu-larly preferred.

The couplers and the developers can be contained in the colorant of the invention either alone or in admixture with one another, the total amount of couplers and developers in the colorant of the invention (based on the total amount of the ready-to-use colorant) in each case being from about 0.005 to 20 weight percent, preferably from about 0.01 to 5 weight percent and particularly from 0.1 to 2.5 weight percent.

The total amount of the developer-coupler combination contained in the colorant of the invention (based on the ready-to-use colorant) is preferably from about 0.01 to 20 weight percent, an amount from about 0.02 to 10 weight percent and particularly from 0.2 to 6 weight percent being particularly preferred. In general, the developers and couplers are used in equimolar amounts; it is not disadvantageous, however, if in this respect the developers are present in a certain excess or deficiency.

Moreover, the colorant of the invention can also contain other dye components, for example 6-amino-2-methylphenol and 2-amino-5-methylphenol, as well as common direct dyes from the group of acidic or basic dyes, triphenylmethane dyes, aromatic nitro dyes, azo dyes and disperse dyes. The colorant of the invention can contain these dye components in an amount from about 0.1 to 4 weight percent (based on the ready-to-use colorant).

Naturally, the couplers and developers and the other dye components, provided they are bases, can also be used in the form of their physiologically compatible salts of organic or inorganic acids, for example hydrochloric acid or sulfuric acid or—if they contain aromatic OH groups—in the form of salts of bases, for example as alkali metal phenoxides.

Furthermore, if the colorant of the invention is a hair colorant, it can contain other common cosmetic additives, for example antioxidants such as ascorbic acid, thioglycolic acid and/or sodium sulfite, as well as perfume oils, complexing agents, wetting agents, emulsifiers, thickeners and hair-care agents.

The formulation form of the colorant of the invention can be, for example, a solution, particularly an aqueous or aqueous-alcoholic solution. The particularly preferred formulation forms, however, are creams, gels or emulsions. Their composition consists of a mixture of the dye components with the additives commonly used for such compositions.

Common additives for solutions, creams, emulsions, or gels are, for example, solvents, for example, water, lower aliphatic alcohols, for example ethanol, propanol or isopropanol, glycerol or glycols such as and 1,2-propylene glycol, moreover wetting agents or emulsifiers from the classes of anionic, cationic, amphoteric or nonionic surface-active substances, such as the fatty alcohol sulfates, ethoxylated fatty alcohol sulfates, alkylsulfonates, alkylbenzenesulfonates, alkyltrimethylammonium salts, alkylbetaines, ethoxylated fatty alcohols, ethoxylated nonylphenols, fatty alkanolamides, ethoxylated fatty esters, furthermore thickeners such as the higher fatty alcohols, starch or cellulose derivatives, petrolatum, paraffin oil and fatty acids and also hair-care agents such as cationic resins, lanolin derivatives, cholesterol, pantothenic acid and betaine. The said constituents are used in amounts usually employed for such purposes, for example the wetting agents and emulsifiers at a concentration from about 0.5 to 30 weight percent, the thickeners in an amount from about 0.1 to 30 weight percent and the hair-care agents at a concentration from about 0.1 to 5.0 weight percent.

Depending on the composition, the colorant of the invention can be weakly acidic, neutral or alkaline. The pH is, in particular, from 6.5 to 11.5. A basic pH is preferably obtained with ammonia, but an organic amine, for example monoethanolamine or triethanolamine, or an inorganic base, for example sodium hydroxide or potassium hydroxide, can also be used. To adjust the pH to an acidic value, an inorganic or organic acid, for example phosphoric acid, acetic acid, citric acid or tartaric acid can be used.

For oxidative dyeing of hair, the afore-described colorant is mixed with an oxidant just before use, and an amount of this mixture sufficient for the hair-coloring treatment, generally about 60 to 200 grams depending on the hair fullness, is applied to the hair.

The oxidant used for developing the hair coloration is mainly hydrogen peroxide or a compound of addition thereof to urea, melamine, sodium borate or sodium carbonate, in the form of a 3 to 12% and preferably a 6% aqueous solution, but also atmospheric oxygen. If a 3 to 6% hydrogen peroxide solution is used as oxidant, the weight ratio of hair colorant to oxidant is from 5:1 to 1:5, preferably from about 2:1 to 1:3 and particularly 1:1. Higher amounts of oxidant are used primarily with higher dye concentrations in the hair colorant or when at the same time more intense bleaching of the hair is intended. The addition of other oxidants, for example sodium persulfate, potassium persulfate or ammonium per-sulfate, is in principle also possible as is the use of atmospheric oxygen as oxidant.

The mixture is allowed to act on the hair at 15 to 50° C. for about 10 to 45 minutes and preferably from 15 to 30 minutes after which the hair is rinsed with water and dried. Following this rinsing, the hair is additionally washed with a shampoo and optionally post-rinsed with a weak organic acid, for example citric acid or tartaric acid. The hair is then dried.

The following examples are intended to explain the subject matter of the invention in greater detail without limiting it to said examples.

### EXAMPLES

In the examples that follow, the following abbreviations are used for the dyes employed:

A: 1,4-diamino-2-methylbenzene hydrosulfate

B: 4,5-diamino-1-(2′hydroxyethyl)-1H-pyrazole hydrosulfate

C: 4-aminophenol

D: 2-(2′-hydroxyethyl)-1,4-diaminobenzene sulfate

E: 1,3-dihydroxybenzene

F: 3-aminophenol

G: 5-amino-2-methylphenol

H: 1-naphthol

I: 1,3-diamino-4-(2′-hydroxyethoxy)benzene hydrochloride

J: N-(2′-hydroxyethyl)-3,4-methylenedioxyaniline hydrochloride

K: 2-amino-6-chloro-4-nitrophenol

L: N-(2′-hydroxyethyl)-2-nitro-4-(trifluoromethyl)aniline

### Examples 1 to 39

Oxidation Hair Colorant, Creamy

| | | |
|---|---|---|
| 22.0 | g | of cetylstearyl alcohol |
| 1.0 | g | of wool wax alcohol |
| 0.3 | g | of cholesterol |
| 6.8 | g | of sodium lauryl alcohol diethylene glycol ether sulfate, |
| | | 28% aqueous solution |
| 0.5 | g | of sodium sulfite, anhydrous |
| 0.1 | g | of disodium ethylenediaminetetraacetate |
| X | g | of dye combination as per Table 1 |
| 8.0 | g | of ammonia, 25% aqueous solution |
| to 100.0 | g | water |

Just before use, 20 g of the above creamy colorant was mixed with 20 g of a 4.5% aqueous hydrogen peroxide solution. The resulting mixture (pH=9.5-10) was apilied to bleached hair and after an exposure time of 30 min at room temperature rinsed out with water. The hair was then washed with a commercial shampoo and dried.

The dye combinations and the coloring results obtained are collected in the following Table 1.

TABLE 1

| Example | | |
|---|---|---|
| No. | Dye Combination | Color |
| 1 | 1.25 mmol A + 1.25 mmol E + 3 mmol L | light green |
| 2 | 1.25 mmol A + 1.25 mmol F + 3 mmol L | light khaki colors |
| 3 | 1.25 mmol A + 1.25 mmol G + 3 mmol L | chocolate brown |
| 4 | 1.25 mmol A + 1.25 mmol H + 3 mmol L | dark khaki colors |
| 5 | 1.25 mmol A + 1.25 mmol I + 3 mmol L | blue-green |
| 6 | 1.25 mmol A + 1.25 mmol J + 3 mmol L | green-yellow |
| 7 | 1.25 mmol B + 1.25 mmol E + 3 mmol L | warm gold |
| 8 | 1.25 mmol B + 1.25 mmol F + 3 mmol L | red-orange |
| 9 | 1.25 mmol B + 1.25 mmol G + 3 mmol L | orange-red |
| 10 | 1.25 mmol B + 1.25 mmol H + 3 mmol L | red |
| 11 | 1.25 mmol B + 1.25 mmol I + 3 mmol L | cold red |
| 12 | 1.25 mmol B + 1.25 mmol J + 3 mmol L | copper red |
| 13 | 1.25 mmol C + 1.25 mmol E + 3 mmol L | fox red |
| 14 | 1.25 mmol C + 1.25 mmol F + 3 mmol L | slightly dull gold |
| 15 | 1.25 mmol C + 1.25 mmol G + 3 mmol L | dull gold |
| 16 | 1.25 mmol C + 1.25 mmol H + 3 mmol L | very dull gold |
| 17 | 1.25 mmol C + 1.25 mmol I + 3 mmol L | lemon-yellow |
| 18 | 1.25 mmol C + 1.25 mmol J + 3 mmol L | gold-blond |
| 19 | 1.25 mmol D + 1.25 mmol E + 3 mmol L | gold-orange |
| 20 | 1.25 mmol D + 1.25 mmol F + 3 mmol L | copper colors |
| 21 | 1.25 mmol D + 1.25 mmol G + 3 mmol L | copper brown |
| 22 | 1.25 mmol D + 1.25 mmol H + 3 mmol L | dull gold |
| 23 | 1.25 mmol D + 1.25 mmol I + 3 mmol L | green |
| 24 | 1.25 mmol D + 1.25 mmol J + 3 mmol L | lemon-green |
| 25 | 1.25 mmol A + 1.25 mmol E + 0.5 mmol K + | gold-blond |
| | 2.4 mmol L | |
| 26 | 1.25 mmol B + 1.25 mmol E + 0.5 mmol K + | copper-gold |
| | 2.4 mmol L | |
| 27 | 1.25 mmol C + 1.25 mmol E + 0.5 mmol K + | corn-yellow |
| | 2.4 mmol L | |
| 28 | 1.25 mmol D + 1.25 mmol E + 0.5 mmol K + | bright gold-blond |
| | 2.4 mmol L | |
| 29 | 1.25 mmol A + 0.21 mmol E + 0.21 mmol F + | dark olive-green |
| | 0.21 mmol G + 0.21 mmol H + 0.21 mmol I + | |
| | 0.21 mmol J + 3 mmol L | |
| 30 | 1.25 mmol B + 0.21 mmol E + 0.21 mmol F + | copper-red |
| | 0.21 mmol G + 0.21 mmol H + 0.21 mmol I + | |
| | 0.21 mmol J + 3 mmol L | |
| 31 | 1.25 mmol C + 0.21 mmol E + 0.21 mmol F + | cognac colors |
| | 0.21 mmol G + 0.21 mmol H + 0.21 mmol I + | |
| | 0.21 mmol J + 3 mmol L | |
| 32 | 1.25 mmol D + 0.21 mmol E + 0.21 mmol F + | bright olive-green |
| | 0.21 mmol G + 0.21 mmol H + 0.21 mmol I + | |
| | 0.21 mmol J + 3 mmol L | |
| 33 | 1.25 mmol A + 0.21 mmol E + 0.21 mmol F + | dull gold-brown |
| | 0.21 mmol G + 0.21 mmol H + 0.21 mmol I + | |
| | 0.21 mmol J + 0.5 mmol K + 2.4 mmol L | |
| 34 | 1.25 mmol B + 0.21 mmol E + 0.21 mmol F + | copper-red |
| | 0.21 mmol G + 0.21 mmol H + 0.21 mmol I + | |
| | 0.21 mmol J + 0.5 mmol K + 2.4 mmol L | |
| 35 | 1.25 mmol C + 0.21 mmol E + 0.21 mmol F + | copper colors |
| | 0.21 mmol G + 0.21 mmol H + 0.21 mmol I + | |
| | 0.21 mmol J + 0.5 mmol K + 2.4 mmol L | |
| 36 | 1.25 mmol D + 0.21 mmol E + 0.21 mmol F + | gold-brown |
| | 0.21 mmol G + 0.21 mmol H + 0.21 mmol I + | |
| | 0.21 mmol J + 0.5 mmol K + 2.4 mmol L | |
| 37 | 0.75 mmol B + 0.64 mmol E + 0.57 mmol G + | orange |
| | 3 mmol L | |
| 38 | 0.75 mmol B + 0.78 mmol J + 3 mmol L | copper gold |
| 39 | 0.37 mmol B + 0.36 mmol D + 0.78 mmol J + | copper beige |
| | 3 mmol L | |

### Example 40

Comparative Tests

| | | |
|---|---|---|
| 22.0 | g | of cetylstearyl alcohol |
| 1.0 | g | of wool wax alcohol |
| 0.3 | g | of cholesterol |
| 6.8 | g | of sodium lauryl alcohol diethylene glycol ether sulfate, |
| | | 28% aqueous solution |
| 0.5 | g | of sodium sulfite, anhydrous |
| 0.1 | g | of disodium ethylenediaminetetraacetate |
| X | g | of dye combination as per Table 2 |
| 8.0 | g | of ammonia, 25% aqueous solution |
| to 100.0 | g | water |

The dye combinations (developer and coupler or direct dye) shown in Table 2 were worked into the above basic creamy hair-dyeing composition:

TABLE 2

| Example | Dye Combination |
|---|---|
| 40 a | 2.3 mmol C + 2.7 mmol E |
| 40 b | 5 mmoI K |
| 40 c | 5 mmol L |

Just before use, 20 g of the above coloring cream was mixed with 20 g of a 4.5% aqueous hydrogen peroxide solution. The resulting mixture (pH=9.5-10) was applied to bleached hair and after an exposure time of 30 minutes at room temperature rinsed with water. The hair was then washed with a commercial shampoo and dried. The same washing and drying procedure was repeated 10 times, and after one, five and ten washings the L-values were measured with a Minolta CR-200 color meter supplied by Minolta.

The L-value indicates brightness (namely, the lower the L-value the higher is the color intensity).

TABLE 3

| | | | Δ L |
|---|---|---|---|
| | | | (Comparison with |
| | Number of | | First Washing |
| Example | Wash Cycles | L* | Cycle) |
| 40 a | 1 | 58.1 | |
| | 5 | 59.5 | −1.4 |
| | 10 | 59.9 | −1.8 |
| 40 b | 1 | 52.8 | |
| | 5 | 59.6 | −6.8 |
| | 10 | 61.6 | −8.8 |
| 40 c | 1 | 84.7 | |
| | 5 | 85.0 | −0.3 |
| | 10 | 85.0 | −0.3 |

The comparative tests show that the wash resistance of the direct dye N-(2′-hydroxyethyl)-2-nitro-4-(trifluoromethyl)aniline according to the invention is substantially better than that of the direct control dye 2-amino-6-chloro-4-nitrophenol not according to the invention

Unless otherwise indicated, all percentages given in the present patent application are by weight.

1-10. (canceled)

11. An agent for coloring keratin fibers, said agent containing 4-(2′-hydroxyethyl)-amino-3-nitro-1-trifluoromethylbenzene, or a salt thereof; at least one developer and at least one coupler.

12. The agent as defined in claim 11, wherein the at least one developer is selected from the group consisting of 1,4-diamino-2-methylbenzene; 4,5-diamino-1-(2′-hydroxyethyl)-1H-pyrazole; 4-aminophenol and 2-(2′-hydroxyethyl)-1,4-diaminobenzene.

13. The agent as defined in claim 11, wherein the at least one developer is selected from the group consisting of 4,5-diamino-1-(2′-hydroxyethyl)-1H-pyrazole and 2-(2′-hydroxyethyl)-1,4-diaminobenzene.

14. The agent as defined in claims 11, wherein the at least one coupler is selected from the group consisting of 1,3-dihydroxybenzene; 3-aminophenol; 5-amino-2-methylphenol; 1-naphthol; 1,3-diamino-4-(2′-hydroxyethoxy)benzene; 5-(2′-hydroxyethylamino)-2-methylphenol and N-(2′-hydroxyethyl)-3,4-methylenedioxyaniline.

15. The agent as defined in claim 11, wherein the at least one coupler is selected from the group consisting of 5-amino-2-methylphenol; 1-naphthol; 1,3-diamino-4-(2′-hydroxyethoxy)benzene and N-(2′-hydroxyethyl)-3,4-methylenedioxyaniline.

16. The agent as defined in claim 11, containing from 0.01 to 5 weight percent, based on a total amount of ready-to-apply colorant, of said 4-(2′-hydroxyethyl)amino-3-nitro-1-trifluoromethylbenzene, or said salt thereof.

17. The agent as defined in claim 11, containing from 0.005 to 20 weight percent, based on a total amount of ready-to-apply colorant, of each of said at least one developer and at least one coupler.

18. The agent as defined in claim 11, containing from 0.01 to 20 weight percent, based on a total amount of ready-to-apply colorant, of a total amount of said at least one developer and said at least one coupler.

19. The agent as defined in claim 11, consisting of a hair colorant.

20. A method for oxidative coloring of hair, said method comprising the steps of:
a) immediately prior to coloring the hair, mixing a colorant composition with an oxidant to form a ready-to-apply colorant;
b) applying the ready-to-apply colorant to the hair;
c) allowing the ready-to-apply colorant applied to the hair in step b) to act on the hair for an acting time of 10 to 45 minutes at 15 to 50° C.; and then
d) rinsing the hair and then drying the hair;
wherein the colorant composition contains 4-(2′-hydroxyethyl)-amino-3-nitro-1-trifluoromethylbenzene, or a salt thereof; at least one developer and at least one coupler.
